(19) United States
(12) Patent Application Publication
(10) Pub. No.: US 2009/0286713 A9
(43) Pub. Date: Nov. 19, 2009

(54) Collagen Receptor I-Domain Binding Modulators

(75) Inventors: Jyrki Heino, Turku (FI); Mark Johnson, Turku (FI); Jarmo Kapyla, Lieto (FI); Anne Marjamaki, Littoinen (FI); Tommi Nyronen, Helsinki (FI); Marika Ojala, Rusko (FI); Olli Pentikainen, Lieto (FI); Liisa Nissinen, Turku (FI)

Correspondence Address: BIRCH STEWART KOLASCH & BIRCH, PO BOX 747, FALLS CHURCH, VA, 22040-0747, US

(73) Assignee: Biotie Therapies Corporation, Turku (FI)

(21) Appl. No.: 12/066,955

(22) PCT Filed: Sep. 15, 2006
(86) PCT No.: PCT/FI06/50396

§ 371 Date: May 2, 2008

(30) Foreign Application Priority Data
Sep. 16, 2005 (FI) 20055496

(51) Int. Cl. A61K 38/02; G06G 7/60; A61P 29/00; A61P 7/02; A61P 35/00

(52) U.S. Cl. 514/2; 703/11

(57) ABSTRACT

The present invention relates to a refined and detailed molecular model of the α2⊕1 integrin I-domain, especially the MIDAS and to the use of such a model for designing novel integrin modulators, especially α2β1 integrin modulators. The present invention further relates to novel α2β1 I-domain modulators, which are of therapeutic potential. The present invention further relates to specific families of small molecule modulators interacting with collagen receptors, tetracyclic polyketides and sulfonamides. The present invention further relates to the use of such modulators for the manufacture of medicaments for thrombosis, inflammation and/or cancer

### TECHNICAL FIELD

The present invention relates to a refined and detailed molecular model of the I-domain, especially the metal ion dependent adhesion site called MIDAS and to the use of such a model for designing novel integrin modulators, especially α2β1 integrin modulators. The present invention further relates to novel α2β1 integrin modulators characterized by the key interactions required by the MIDAS amino acid residues, which modulators modulate integrin I-domain interactions, especially collagen binding and function, and which are of therapeutic potential. The present invention further relates to specific families of small molecule modulators interacting with collagen receptors, tetracyclic polyketides and sulfonamides. The present invention further relates to the use of such modulators for the manufacture of medicaments for thrombosis, vascular diseases, inflammation and/or cancer.

### BACKGROUND OF THE INVENTION

The integrins are a large family of cell adhesion receptors, which mediate anchoring of all human cells to the surrounding extracellular matrix. In addition integrins participate in various other cellular functions, including cell division, differentiation, migration and survival. The human integrin gene family contains 18 alpha integrin genes and 8 beta integrin genes, which encode the corresponding alpha and beta subunits. One alpha and one beta subunit is needed for each functional cell surface receptor. Thus, 24 different alpha-beta combinations exist on human cells. Nine of the alpha subunits contain a specific “inserted” I-domain, which is responsible for ligand recognition and binding. Four of the α I-domain containing integrin subunits, namely α1, α2, α10 and α11, are the main cellular receptors of collagens. Each one of these four alpha subunits form a heterodimer with the β1 subunit, which also contains an I-like domain containing another MIDAS (Springer and Wang, 2004). Thus the collagen receptor integrins are α1β1, α2β1 , α10β1 and α11β1 (Reviewed in White et al., Int J Biochem Cell Biol, 2004, 36:1405-1410). Collagens are the largest family of extracellular matrix proteins, composed of at least 27 different collagen subtypes (collagens I-XXVII).

Integrin α2β1 is expressed on epithelial cells, platelets, inflammatory cells and many mesenchymal cells, including endothelial cell, fibroblasts, osteoblasts and chondroblasts (Reviewed in White et al., supra). Epidemiological evidence connect high expression levels of α2β1 on platelets to increased risk of myocardial infarction and cerebrovascular stroke (Santoso et al., Blood, 1999, Carlsson et al., Blood. 1999, 93:3583-3586), diabetic retinopathy (Matsubara et al., Blood. 2000, 95:1560-1564) and retinal vein occlusion (Dodson et al., Eye. 2003, 17:772-777). Evidence from animal models supports the proposed role of α2β1 in thrombosis. Integrin α2β1 is also over-expressed in cancers such as invasive prostate cancer, melanoma, pancreatic cancer, gastric cancer and ovary cancer. These observations connect α2β1 integrin to cancer invasion and metastasis. Moreover, cancer-related angiogenesis can be partially inhibited by anti-α2 function blocking antibodies (Senger et al., Proc. Natl. Acad. Sci. U.S.A., 1997, 94:13612-13617). Finally, leukocytes are partially dependent on α2β1 function during inflammatory process (de Fougerolles et al., J. Clin. Invest., 2000, 105:721-729). Based on the tissue distribution and experimental evidence α1β1 integrin may be important in inflammation, fibrosis, bone fracture healing and cancer angiogenesis (White et al., supra), while all four collagen receptor integrins may participate in the regulation of bone and cartilage metabolism.

The strong evidence indicating the involvement of collagen receptors in various pathological processes has made them potential targets of drug development. Function blocking antibodies against α1 or α2 subunits have been effective in several animal models including models for inflammatory diseases and cancer angiogenesis. Synthetic peptide inhibitors as well as snake venom peptides blocking the function of α1β1 and α2β1 have been described. (Eble, Curr Pharm Design 2005, 11:867-880). International Patent Publication WO 99/02551 discloses one small molecule drug candidate that regulates the expression of α2β1 but it is not actually binding to the integrin.

The collagen binding α I-domains play critical role in rational drug design targeted to the collagen receptors. The mechanism of α2 I-domain binding to one high affinity motif in collagen I is known. However, α I-domains contain multiple other sites that can be potentially interesting for drug development.

A structure of the α2 I-domain in its unligated, “inactive” (“closed”) form has been described by Emsley et al. in J. Biol. Chem, 1997, 272: 28512-7. The key feature of I-domains and the related vWf A-domains is that they contain a characteristic assembly of five parallel and one anti-parallel beta-strand(s), which form the stable platform of the structure. Another crucial feature of I-domains is that they possess an amino acid motif having the sequence DxSxS, where x represents any amino acid. These three amino acids, D151, S153 and S155 are present in the N-terminal loop arising from the first beta strand of the α2 I-domain. These, along with other oxygen-containing residues in nearby peptide loops, co-ordinate the metal ion and constitute the metal ion dependent adhesion site (MIDAS).

International patent publication WO 01/73444 describes the crystal structure of a collagen mimetic triple-helix peptide in complex with integrin α2 I-domain. This publication discloses that in this active (“open”) conformation the metal ion coordinates to Thr221 instead of Asp254, as described in the 1997 inactive structure. In addition to this change in coordinates, WO 01/7344 discloses, that the C-helix in unwound and there is an additional wind in the next helix. This is so far the best approximation of the structure of the I-domain/ collagen complex.

To the best of our knowledge to date there are no known small molecular inhibitors that have been shown to bind to the MIDAS of collagen receptor integrin α2β1. The surface of the collagen binding site of the integrin MIDAS is so large that it is not possible to design small (size <600 g/mol) molecules whose structure would physically cover the whole site. There is thus an existing need for improved models of the (integrin α2β1 I-domain) MIDAS and methods to study small molecule binding to enable design of novel small molecules, which will modulate collagen interactions with integrin α2β1 as desired for drug discovery.

### BRIEF DESCRIPTION OF THE DRAWINGS

FIG. 1 shows the docking of the molecular core structure and key intermolecular interactions of tetracyclic compounds inside the I-domain MIDAS.

FIGS. 2A and 2B show the “open” (black) and “closed” (grey) conformations of α2 I-domain. Superposition is based on two serine residues (153 and 155; ball-and-stick) co-ordinated to the magnesium ion (black sphere). In “open” conformation the Thr221 co-ordinates to the metal ion, while in “closed” conformation this interaction is absent. FIG. 2A above MIDAS and FIG. 2B side view.

FIG. 3 The position of Tyr285 at C-helix stabilizes the binding conformation of inhibitors ligands (shown as a line-model for all docked tetracylic polyketides ligands).

FIG. 4 Positions of the key water molecules inside the integrin α2 I-domain. The MIDAS amino acids derived from the results of the docking simulations are coloured black (within 4 Å from the docked tetracyclic polyketides), grey (distance 4-8 Å from the docked tetracyclic polyketides) or white (over 8 Å from docked tetracyclic polyketides).

FIG. 5 The shape and the volume occupied by an ensemble of small molecule modulators of collagen binding in α2 I-domain MIDAS. The MIDAS amino acids derived from the results of the docking simulations are coloured black (within 4 Å from the docked tetracyclic polyketides), grey (distance 4-8 Å from the docked tetracyclic polyketides) or white (over 8 Å from docked tetracyclic polyketides).

FIG. 6A shows the dose dependent effect of tetracyclic polyketide L3015 on α2 I-domain (200 ng) binding to type I collagen.

FIG. 6B shows the effect of tetracyclic polyketide L3015 on binding of α1I and α2 I-domains (800 ng) to collagen types I and IV.

FIG. 7A shows the effect of lovastatin on binding of α1I and α2 I-domains to type I collagen.

FIG. 7B shows the effect of tetracyclic polyketide L3015 on binding of α2 I-domain to RKK-peptide (about 0.5 mM).

FIG. 8A shows the effect of tetracyclic polyketides L3007, L3008, and L3009 on the binding of α2 I-domain (800 ng) to type I collagen.

FIG. 8B shows the binding of α2 I-domain (800 ng) to type I collagen as a function of tetracyclic polyketide L3009 concentration.

FIG. 9 shows the inhibition of the binding of α1I, α2I, α10I, and α11 I-domains to the type I collagen by tetracyclic polyketide L3009.

FIG. 10A shows the dose dependent inhibition of CHO-α2 cell adhesion to collagen type I by tetracyclic polyketide L3009 and FIG. 10B by sulphonamide derivative compound 434.

FIG. 11A The structure of the α2 I-domain showing the preferred position of the tetracyclic small molecular structure present in compounds reported in this work in the MIDAS.

FIG. 11B The arrangement of amino acids in the vicinity of potential I-domain ligands in the closed form (non-collagen binding) of the I-domain. Key residues within 4 Å radius are shown with black, residues within 4-8 Å with grey and residues within 8-12 Å with white.

FIG. 12 shows that compound 434 increases the closure time of blood.

### BRIEF DESCRIPTION OF THE INVENTION

The present invention relates to a refined in silico model of the MIDAS of an integrin I-domain, characterized by the amino acid coordinates shown in Table 1, especially amino acid coordinates Asp151, Ser153, Ser155, Thr221, Asp254, Tyr285, Leu286 and Leu296 and amino acid coordinates Asn154, Gly218, Asp219, Gly255, Glu256, Asn289, Leu291 and Asp292. Furthermore the invention relates to a model characterized by key water molecules W514, W699, W701, W700, W668, W597, W644 and W506.

The invention also relates to a method of identifying potential modulators of an I-domain-containing integrin using said model to design or select potential modulators.

The present invention further relates to a method of identifying compounds modulating an α2β1 integrin, preferably α2β1 integrin inhibitors. In said method an algorithm for 3-dimensional molecular modelling is applied to the atomic coordinates of an I-domain-containing integrin to determine the spatial coordinates of the MIDAS of a said integrin; and stored spatial coordinates of a set of candidate compounds is virtually screened in silico against said spatial coordinates. Based on this comparison compounds that can bind to the MIDAS of said integrin are identified. Preferably such compounds are integrin inhibitors.

The invention further relates to novel modulators of I-domain-containing integrin, identified or obtained by the method according to the present invention. Integrin modulators according to the present invention are characterized by the key interactions required by the MIDAS amino acid residues, including hydrogen bond donor or acceptor, hydrophobic, hydrogen bond donor and metal ion interactions.

The present invention further relates to novel integrin inhibitors, such as tetracyclic polyketides and sulphonamide derivatives.

The present invention further relates to the use of modulators according to the present invention, preferably to the use of inhibitors for the manufacture of a pharmaceutical composition for the treatment of thrombosis, cancer, fibrosis or inflammation.

Furthermore the present invention relates to a method of treating a thrombosis, vascular diseases, cancer, fibrosis or inflammation by administering an effective amount of an inhibitor according to the present invention.

### DETAILED DESCRIPTION OF THE INVENTION

The present invention relates to a refined and detailed molecular model of the I-domain, especially the MIDAS, in complex with new modulators and to the use of such molecular models for designing novel integrin small molecule modulators, especially α2β1 integrin modulators. Such small molecule integrin modulators bind to integrins according to a binding mechanism that is different from the currently known binding mechanism of a collagen mimetic peptide.

The present invention further relates to the atomic details of the molecular model of the metal ion dependent adhesion site (MIDAS) of the I-domain and the interactions between the binding site atoms and small molecule modulators binding to the site. More specifically, the present invention describes the critical amino acids, the atoms of the peptide main chain and the water molecules, which participate in the complex formation between the αI-domain and the modulators, such as synthetic tetracyclic polyketide and sulphonamide integrin modulators. The tetracyclic polyketide compounds found with the help of structure-based small molecule design are experimentally shown to bind to α2 I-domain.

Furthermore the present invention relates to structure-based rules of designing α2 integrin binding novel small molecules based on the α I-domain structure model derived from publicly available X-Ray data.

The rules of small molecule binding to the MIDAS amino acids reported in this invention are applicable to the binding of other chemical entities than tetracyclic polyketides or sulfonamides as well, as long as they satisfy the reported intermolecular interactions found to be critical for ligands to bind to the I-domain.

It is further shown that the methods of the present invention are useful for designing and screening inhibitors that bind to collagen receptor integrins α1β1, α10β1 and α11β1 in addition to α2β1 integrins.

The present invention further relates to novel α I-domain modulators characterized by the key interactions required by the MIDAS amino acid residues, including hydrogen bond donor or acceptor (HBDA), hydrophobic (HYD), hydrogen bond donor (HBD) and metal ion (Mg) interactions. The modulators according to the present invention may also interact with or replace water molecules present in the MIDAS.

Integrin modulators according to the present invention include direct I-domain MIDAS-binding modulators and allosteric I-like domain MIDAS binding modulators. Such modulators are preferably inhibitors.

The present invention thus provides novel integrin-inhibitors, such as tetracyclic polyketides and sulphonamide derivatives

The present invention provides the use of such integrin-modulators for the manufacture of a medicament for use in the treatment of diseases related to thrombosis, cancer, fibrosis and inflammation.

### Details of Structural Features of the I-Domain

The present structural knowledge of the α2 integrin I-domain is based on the above cited publications describing two static structures of the I-domain, the closed and the open forms. In reality, the I-domain, and especially the different parts of the MIDAS, are mobile. The I-domain changes its conformation in response to the molecular environment in the cell. Different conformations can be induced by other molecules binding to the MIDAS. The design of small molecules that compete with biological molecules in binding, thus being able to modulate interactions of biologically significant molecular entities with the MIDAS, requires detailed information of the dynamics of the receptor-ligand interaction that cannot be derived merely from the two static receptor models.

The two published structures may be compared to two “photographs” of the mobile domain. In the present invention the information derived from the crystal structures has been extended by molecular modelling, and so called ensemble-models have been created, wherein the possibilities of the I-domain (and especially the MIDAS) to conform to the structures of binding ligands has been investigated using Bodil Modeling Environment (Lehtonen et. al. 2004). Furthermore the conformational space and the receptor-induced conformational changes of the ligands have been investigated with flexible docking study (program FlexX in Sybyl, Tripos Inc.).

The I-domain interaction with collagen has been previously studied by mutation experiments and the sites constituting the collagen contact with α2 I-domain have been shown to be Asn154, binding the β A-chain and alpha helix 1; Asp219 and Leu200 binding alpha helixes 3 and 4; Glu256, His258 binding β D-chain and alpha helix 5; Tyr285, Asn289, Leu291, Asn295 and Lys298 binding the C-helix, alpha helix 6, β C-chain and alpha helix 6. It is also known that an Ala mutation in Glu256 and Asn295 do not induce changes in collagen binding.

Mutations affecting Asp151, Ser153, Thr221 and Asp254 are known to cause changes in collagen binding. The effect is mainly due to the fact that these amino acids bind to the metal ion of the I-domain, which is essential for the collagen binding.

International patent publication WO 01/73444 discloses that a trimeric collagen mimetic GFOGER-peptide binding to α2 I-domain is affected by the following amino acids: in the middle strand of helix 1 glutamate coordinates to the metal and Arg forms a salt bridge with Asp219, while phenylalanine is situated between Gln215 and Asn154; in the trailing strand of the outmost helix 2 phenylalanine is in contact with Tyr157, Leu286 and arginine is close to Glu256, but does not form a salt bridge in the crystal structure; in the main chain of helix 3 there is a hydrogen bond between Asn154 and Tyr157 forming a contact loop 1; and there is a His258 in loop 3.

When the collagen mimetic peptide binds to the MIDAS, there are clear conformational changes. The Mg2+ metal coordination changes and the C-helix of the I-domain moves away form the collagen when it coordinates to the metal. The changes in the structure as a result of this movement have a structural impact all the way on the opposite pole of the I-domain.

As described in international patent publication WO 01/73444 the collagen “pushes” the metal towards amino acid Thr221 when the I-domain changes from the closed form to the open form. The loop in the MIDAS follows the movement. The metal coordination at Ser153 as well as at Ser155 is unchanged, but the Asp254 metal bond is broken. The Gly255 peptide bond is rotated 180 degrees and moves away form the metal. Glu256 forms a bond to the metal through water. Tyr175 and His258 sink into the collagen trimeric helix strands, at least in connection to the collagen mimetic peptide.

As a result of this helix 7 changes radically, the MIDAS C-helix unwinds and there is a new coil formed in alpha helix 6. The most important conformational change from the vantage point of complex formation is that collagen glutamate moves towards the metal and coordinates with it. Before the collagen can form contact to the I-domain MIDAS metal, it has to overcome a steric hindrance by the Tyr285 side chain. This amino acid is located in the C-helix of the I-domain.

The conformational changes in the α I-domain lead to another conformational change in the whole alpha-beta heterodimer, which in turn leads to activation of intracellular signalling pathways, possibly because of the cytoplasmic domains of the α- and β-subunits moving further away from each other.

During modelling and refinement of the existing models the following observations for modulator design were made:

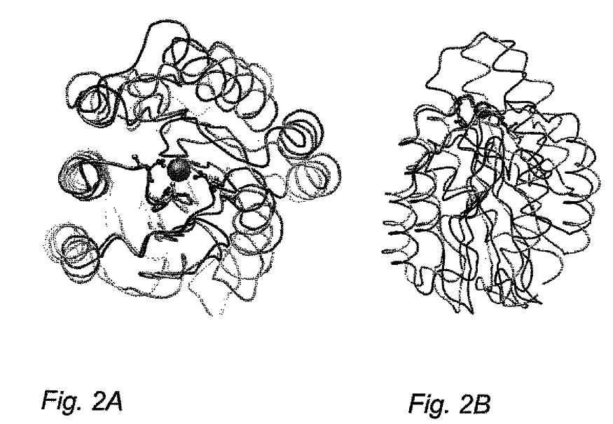

The biological role of the C-helix in the I-domain may reside in the inhibition of collagen binding to the metal. It is important to take this fact into consideration when designing small molecule modulators of collagen binding. Collagen is inhibited from binding the I-domain in the closed form of the receptor by the C helix conformation. Therefore, modulator features that can further stabilize the C-helix in the closed form are important properties when designing novel small molecule collagen binding modulators. Modulators designed with this property in mind inhibit collagen binding, as is shown by our experiments. FIG. 2 depicts in grey colour the closed form of the I-domain and in black colour the open form with bound collagen mimetic GFOGER-peptide (collagen peptide not shown for clarity). When designing collagen binding inhibiting modulators the binding of the modulators should stabilize the closed form of the I-domain, which would inhibit the collagen binding.

General Observations and Rules for Modulator Design Arising from the MIDAS Structure

In contrast to the interpretation of the earlier reported structural changes upon the binding of the collagen mimetic, we have also found new features of the positions and distances (geometry) of the key MIDAS amino acids serine and threonine (Ser153, Ser155 and Thr221), which are important for the design of novel collagen binding modulators. The interpretation of the changes in the rigid protein coordinates is dependent of the method used for superimposing the coordinates and thus affects the interpretation of the superposition. Instead of using the entire protein structure as a measure of superimposition, the present modelling focused on the structure of the MIDAS in the superimposition. Surprisingly, this leads to novel interpretation of the structural changes that take place upon binding of the collagen mimetic GFOGER-peptide. In the present invention, the superposition of the open and closed forms is made using the coordinates of the key amino-acid side chains of Ser153 (and Ser55). Then, in contrast to the earlier interpretations, the metal ion of the MIDAS then remains close to its original position instead of moving. Rather, the main chains of the protein surrounding the metal reorganize to reach closer to each other in the open conformation of the I-domain compared to the closed form.

Changes take place in the Mg2+ metal coordination. Thr221 coordinates to the metal and one coordinated water molecule is removed. The observation from this alternate superposition is that at the same time in the open conformation the main chains of the protein surrounding the metal form a new contact with each other. Ser153 and Thr221 are closer to each other in the open form than in the closed form. Features of the modulators that are aimed to stabilize the closed form should thus emphasize the stabilization of the position of the Thr221 in the closed form in such a way that it continues to coordinate to the metal through a water molecule. This will prevent Thr221 from moving closer to metal ion, and assuming the metal coordination typical for the open form of the I-domain. The crystal water W597 is tightly bound to the receptor and Thr221 in the closed form of the I-domain. A modulator may act e.g. by capturing this crystal water to vicinity of Thr221 by accepting a hydrogen bond from W597 (described in detail further on). In the open conformation the water W597 is removed. A Thr221-crystal water-metal stabilizing modulator thus stabilizes the closed form and can inhibit collagen binding.

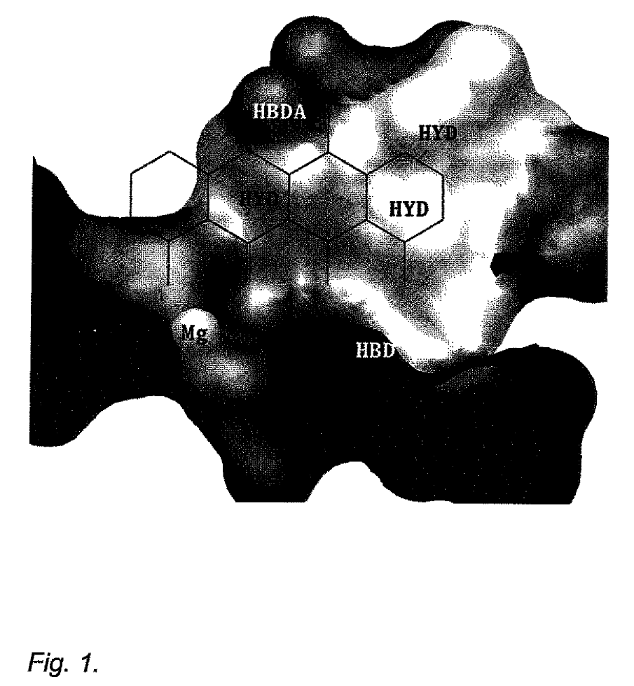

It has further been found, that specific features of the amino acids in the wall of the binding pocket in the C-helix of the I-domain are useful for structural design of modulators. If the modulator interacts constructively with the C-helix (e.g. hydrophobic face of the C-helix, FIG. 1) in the closed form of the I-domain, it stabilizes of the structure of the C-helix, resulting in further modulation of collagen binding.

In FIG. 1 it is shown that the hydrophobic face (white area) of the MIDAS ligand binding cavity is preferably buried by the binding ligands. The ligand position can be stabilized by the key interactions with magnesium ion and main-chain amino group of Glu256 (HBD) and hydroxyl group of Tyr285. These interactions are the key stabilizing interactions to maintain the receptor in “closed” conformation thus, forming the basic pharmacophore for new ligand discovery.

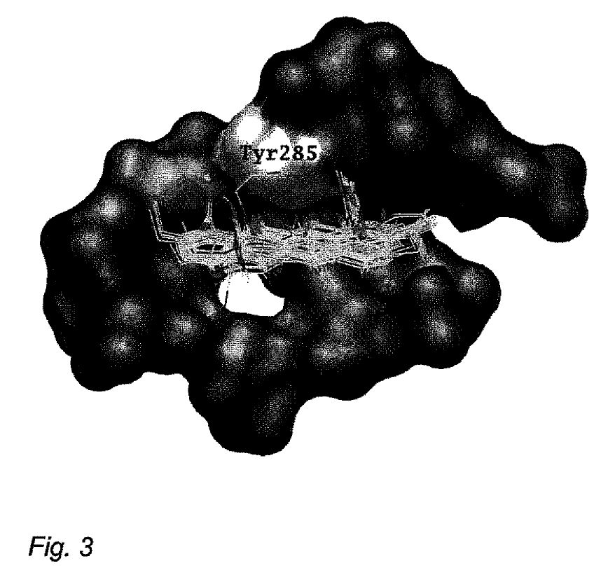

For example, tetracyclic polyketides can form an aromatic-aromatic (pi-pi) interaction with Tyr285 in the C-helix. In FIG. 3 it is shown that the position of Tyr285 at C-helix stabilizes the binding conformation of ligands (shown as a line-model for all docked ligands). In simulation experiments, the hydrogen bond acceptors in this class of modulators were also shown to form hydrogen bonds with the hydroxyl of tyrosine, and between the hydroxyl and carbonyl groups of the modulators.

In the simulations the amino acids Leu286 and Leu29l of the C-helix and helix 6 were shown to form hydrophobic interactions with the hydrophobic isopropyl-ethyl groups of the modulators and the aromatic end groups of the structures. This interaction was also found to be important for the C-helix stabilization.

Specific Interactions between the α2 I-Domain and Potential Modulators

The present invention provides a general three-dimensional form of the MIDAS of the closed form of the I-domain. The shape of the MIDAS is important for the design of modulators. The matching of the shapes of the protein-modulator of the closed form also limits the introduction/removal of new chemical groups that are added to improve e.g. pharmacological properties like solubility, absorption or metabolism. These improvements should not excessively disturb the binding affinity of the compound, which is the primary requirement for successful lead compounds.

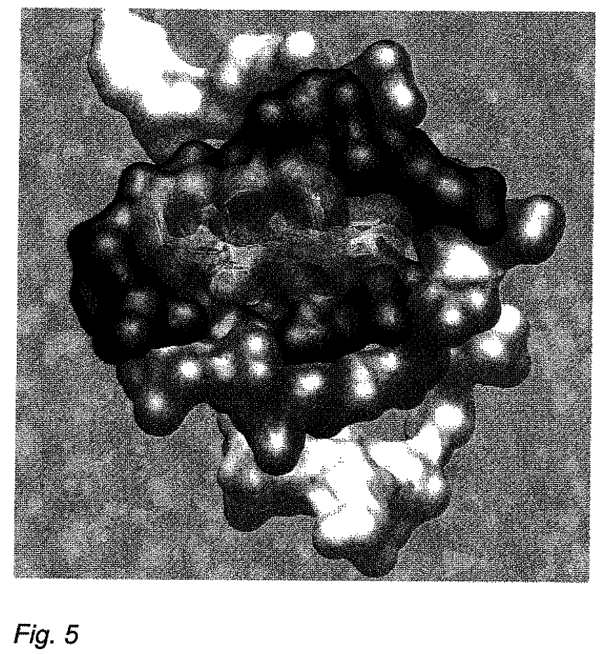

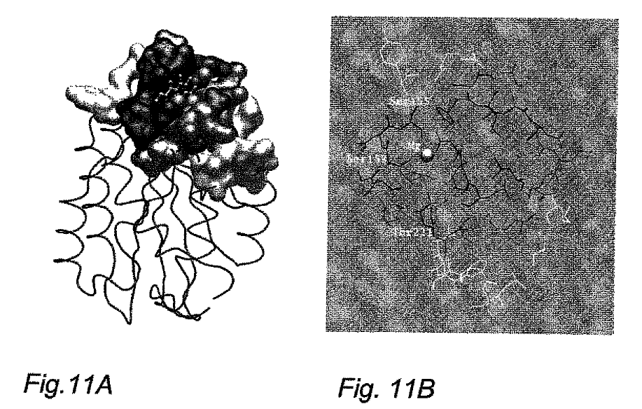

Table 1 and FIGS. 5 and 11B provide a detailed description of the amino acids of the α2 I-domain binding site, the atoms of the main chain and the crystal waters, which all are structurally important when designing modulators interacting with the α2 I-domain.

Based on the docking simulation experiments using Bodil Modeling Environment (Lehtonen et al., al. http://www.abo.fi/fak/mnf/bkf/research/johnson/bodil.html; 2004) and Sybyl (6.9.1. St. Louis, Mo., USA, Tripos Inc.) the following amino acids are identified in Table 1, wherein the distance of each amino acid in relation to the modulators is listed.

TABLE 1

| | L1 < 4 Å | L2 < 8 Å | L2 > 8 Å |
|---|---|---|---|
| ASP151 | X | | |
| SER153 | X | | |
| ASN154 | X | | |
| SER155 | X | | |
| TYR157 | | | X |
| ALA188 | | X | |
| GLY218 | X | | |
| ASP219 | X | | |
| LEU220 | | X | |
| THR221 | X | | |
| THR223 | | | X |
| PHE224 | | | X |
| THR253 | | X | |
| ASP254 | X | | |
| GLY255 | X | | |
| GLU256 | X | | |
| SER257 | | X | |
| HIS258 | | X | |
| ASP259 | | | X |
| GLY260 | | | X |
| SER261 | | | X |
| LEU263 | | | X |
| TYR285 | X | | |
| LEU286 | X | | |
| ARG288 | | | X |
| ASN289 | X | | |
| ALA290 | | X | |
| LEU291 | X | | |
| ASP292 | X | | |
| THR293 | | X | |
| LYS294 | | X | |
| ASN295 | | X | |
| LEU296 | X | | |
| LYS298 | | | X |
| GLU299 | | X | |
| ALA302 | | | X |

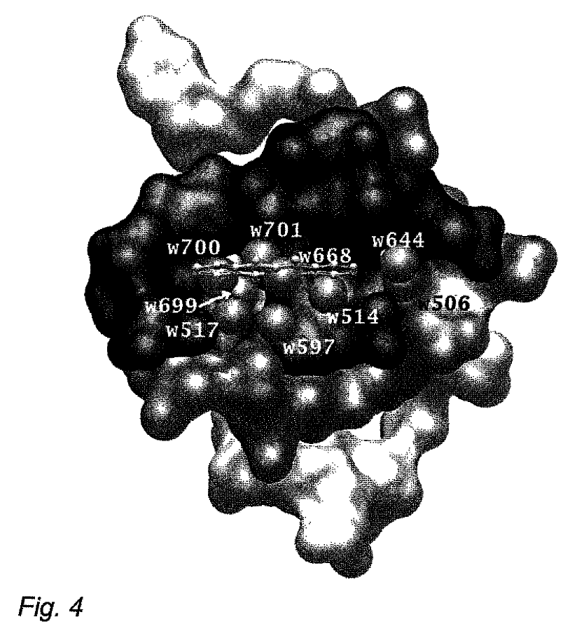

Table 1 lists the amino acids that provide important interactions according to the molecular docking experiments using tetracyclic polyketides. The MIDAS amino acids have been divided into three layers, which correspond to white, grey and black colours in FIG. 4 and FIG. 5. Layer 1 lists MIDAS amino acids within 4 Å of the docked ligands and is depicted in black colour in FIG. 4 and FIG. 5; Layer 2 (Grey in FIGS. 4 and 5): MIDAS amino acids within distance of 4-8 Å from the docked ligands; and Layer 3 (white in FIGS. 4 and 5): MIDAS amino acids over 8 Å from the docked ligands. The most important binding site amino-acid side chain interactions are those directly interacting with the ligand structures (Layer 1, black in FIGS. 4 and 5). The other layers can also influence the binding of the ligands to the MIDAS by “pushing” and otherwise influencing Layer 1 amino acids. The binding site is flexible, thus the amino acids in different layers can change their position or orientation dynamically in response to the binding ligands. Ligands can induce different receptor conformations. The focus of the present ligand design strategy concentrates on being able to modulate binding of biologically important molecules (collagen) to the MIDAS. Therefore, all three layers are important for designing new ligands.

### Further Potential Interactions Stabilizing the Closed Form

Interactions between the modulators/ligands and the protein main chain atoms and functional groups are important for the structural design process. Main-chain atoms are less mobile than the amino-acid side chain atoms, and thus can effectively be used to anchor the modulator to the protein with constructive interactions. The following provides a list of main chain interactions that can be used when designing structures of novel small molecule collagen binding modulators. Formation of most of the interactions requires replacement of a crystal water molecule from the MIDAS. In the list, the following definitions are used: —NH—, to define a main chain amino group, and O═, to define a main chain carbonyl group. In the numbering of the main chain interactions we have used numbering from the closed conformation of I-domain published in the PDB-structure PDB: 1aox.
Ser155, —NH—, can donate one hydrogen bond to the modulator, e.g. hydrogen bond donor (1*HBD)
Gly218, O═, one free lone pair free to accept a hydrogen bond from the modulator, e.g. hydrogen bond acceptor(1*HBA)
Asp219, O═, 1*HBA, one lonepair can accept a hydrogen bond. Ligand access to the second lone pair of Asp219 is sterically blocked by the imidazole ring of key amino acid His258
Asp254, O═, 1*HBA, second position occupied by crystal water W701, otherwise the position is buried and not likely accessible by modulators
Glu256, is one of the key contacts for binding modulators according to modelling
Further definition: Glu256,—NH—, 1*HBD, change in the orientation of the Glu256 amino-acid side chain can cause it to turn and form interaction with e.g. OH-group from the modulator. It is geometrically and physically possible for the modulator OH-group to simultaneously form contact with Glu256 —NH—. Crystal water also resides close-by, which can further stabilize the relocation of the Glu256 amino-acid side chain
Ser257, O═, 2*HBA, crystal water W650 is the nearest possible interaction with this functional group
Gly260, —NH—, 1*HBD, weak interaction with side chain oxygen of Ser257, buried
Asp292, O═, optionally 2*HBA, —NH— 1*HBD, in the closed form the amino acid is located in a hydrophobic pocket and is hydrogen bonded to W506. Replacement of the weakly bound water with proper functional group from the modulator is recommended. This interaction is further defined with crystal waters.
Asn295, —NH—, 1*HBD, buried, less likely to be accessible by modulators
Leu296, —NH—, 1*HBD, buried, less likely to be accessible by modulators

### Crystal Water Molecules

Substituting the water molecules with corresponding modulator substituents (e.g., —OH) is one option for improving the binding of the modulators. It is also shown that the water molecules play an active role in the collagen binding event. Water molecules can have important roles as mediators of key intermolecular interactions, as is described in detail herein. The numbering of the crystal waters corresponds to the numbering in the closed conformation of I-domain reported in the PDB-structure PDB: 1aox.

During the simulation experiments it was further noted that the modulators that stabilize the correct crystal water molecules may have functional roles for the stabilization of the closed form, as the crystal water molecules form hydrogen bonds with several atoms with the amino acids that change their position when the MIDAS reorganizes towards the open form. Positions of the key water molecules inside the α2β1 integrin I-domain are shown in FIG. 4.

Amino acid Glu256 is in the closed form coordinated to water molecule W514, and the tested/designed α2 I-domain tetracyclic and sulphonamide modulators are able to replace its OH-group.

The waters coordinated to the metal are W699, W701 and W700. Water W699 also stabilizes the position of threonine Thr221. A binding ligand may stabilize this water position indirectly by closing its exit route, and thus physically prevent Thr221 from assuming its metal-coordinated position in the open form. Based on the analysis the other lone pair of water W699 seems to be unsaturated in the closed conformation and subject to hydrogen bond donor interaction from the modulator.

Water W700, which is likely to be replaced by many modulators upon binding, is coordinated to the amino group of the main chain of Ser155 and to the metal. The main chain amino group of Ser155 is a possible site for donating a strong hydrogen bond for the modulator. The water molecule is replaced in upon collagen mimetic binding in the open form of the I-domain. When designing modulators, two approaches may be chosen: the water may be replaced in order to improve the binding of the modulator by introducing a hydrogen bond acceptor to this position, or the water may be retained by the modulator, if the water is important for the stability of the closed form.

Water W668 is coordinated only to other water molecules and modulator binding is normally replacing it from the MIDAS.

Water W597 is hydrogen bonded to three sites. Water W668, and the Glu256 O═ of the main chain. Furthermore the water accepts one hydrogen bond from Thr211. This water molecule clearly stabilizes the position of threonine Thr221. In modulator design this water is important for stabilizing the closed conformation and thus is suggested to have key functionality with respect to the modulation of collagen binding. Water W597 is in a good position for donating a hydrogen bond to the modulator, whereby it will become locked in its position by three hydrogen bonds.

Water W644 and W506 are close to Asp 292. These water molecules donate hydrogen bonds to the carboxyl group of Asp292. Water W506 is located in a groove, which is basically hydrophobic, except in the vicinity of the oxygen of the main chain of Asp292. The groove is also mentioned above, and may be defined by the main chain of the protein (amino acids 255 and 256) on the MIDAS; Leu286 (hydrophobic side chain); Asp292 (O═ and —NH—, C-beta carbon of the main chain); Thr293 (main chain, plane of the peptide bond); Lys294 (peptide bond to threonine); Asn295 (main chain —NH—, c-beta carbon, may turn towards the groove); Leu296 (main chain —NH—, c beta carbon); and Glu256 (carboxylate group may turn towards the modulator).

### Characterization of Potential I-Domain Binding Modulators

The chemical structure of the I-domain binding modulators can vary considerably, but they all have to possess structural and chemical similarities in the contacts they form with the above described amino acids of the binding site, with the atoms of the main chain and the crystal water molecules.

It is also important to take into account the general structure of the small molecules binding site, as modulators that may not conform to the structure of the I-domain in certain energy windows cannot bind to the I-domain.

Based on the simulations on tetracyclic polyketides the general shape and the volume that I-domain targeting modulators may occupy is presented in FIG. 5. The MIDAS amino acids have been divided into three layers, which correspond to white, grey and black colours in FIG. 4 and FIG. 5. The layers indicate the distance of the amino acids from the docked ligand (see also Table 1). The most important binding site amino-acid side chain interactions are those directly interacting with the ligand structures (Layer 1, black in FIGS. 4 and 5). The other layers can also influence the binding of the ligands to the MIDAS by “pushing” and otherwise influencing Layer 1 amino acids. The binding site is flexible, thus the amino acids in different layers can change their position or orientation dynamically in response to the binding ligands. Ligands can induce different receptor conformations. The focus of the present ligand design strategy concentrates on being able to modulate binding of biologically important molecules (collagen) to the MIDAS. Therefore, all three layers are important for designing new ligands.

In FIG. 1 it is shown that the hydrophobic face of the ligand binding cavity is buried by the ligands. In addition, the ligand position is stabilized by the key interactions with magnesium ion and main-chain amino group of Glu256 (HBD) and hydroxyl group of Tyr285. These interactions are the key stabilizing interactions to maintain the receptor in “closed” conformation thus, forming the basic pharmacophore for ligand discovery.

The possible compounds that could modulate an I-domain-containing integrin function were identified by using virtual screening technique combined with the pharmacophore model based on the three-dimensional coordinates of integrin I-domain MIDAS. The pharmacophore model contained the key interaction sites, described above, for modulator binding.

Based on the refined computer aided molecular model described above, the present invention provides molecules that fit in the canyon in α2 I-domain surface, which harbours the MIDAS. More specifically, it provides in silico designed and wet lab tested compounds that interact with Mg, bind with good affinity and prevent collagen binding.

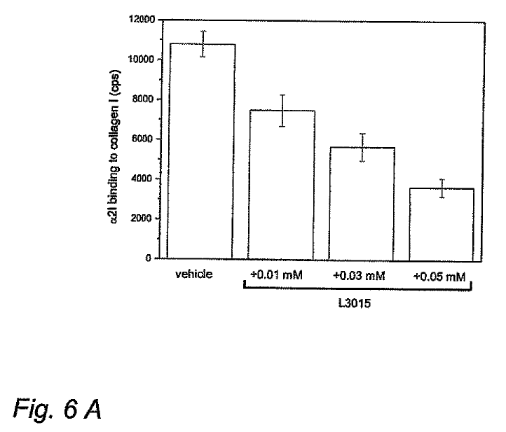

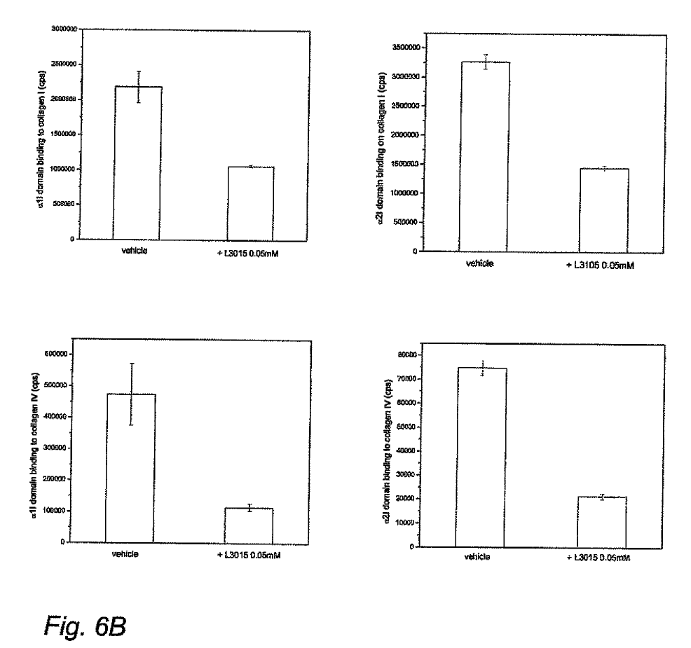

Streptomyces-derived aromatic polyketides that are flat tetracyclic compounds containing suitable oxygen atoms possibly interacting with MIDAS were chosen as a suitable library for screening. Compounds modelled to fit the canyon and the oxygen in the second ring were assumed to interact with Mg ion in MIDAS (FIG. 6). The screening of the compounds in a solid phase α2 I-domain binding assay confirmed the tested hypothesis. The fact that collagen I binding by all four α I-domain was blocked by these compounds indicated that they have a common binding mechanism.

The in silico model was further utilised to identify novel collagen receptor modulators. Sulphonamide derivates are an example of a compounds that were identified using the in silico method according to the present invention, and which fulfil the above criteria. Such compounds were further verified to be collagen receptor modulators using the assays described herein.

Sulphonamide derivatives identified by the methods of the present invention may be described by formula (I),

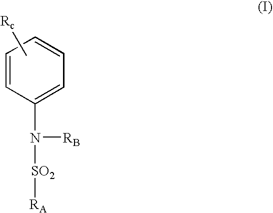

where
R_{C} is selected from a group consisting of dialkylamino, NO₂, CN, aminocarbonyl, monoalkylaminocarbonyl, dialkylaminocarbonyl, alkanoyl, oxazol-2-yl, oxazolylaminocarbonyl, aryl, aroyl, aryl-CH(OH)—, arylaminocarbonyl, furanyl, where the aryl, aroyl and furanyl moieties may be substituted, guanidinyl-(CH₂)_{z}-N(R′)—, Het-(CH₂)_{z}-N(R′)—, Het—CO—N(R′)—, Het—CH(OH)— and Het—CO—, where Het is an optionally substituted 4-6-membered heterocyclic ring containing one or more heteroatoms selected from N, O and S, R′ is hydrogen or alkyl, and z is an integer 1 to 5;
R_{A} is a group having the formula

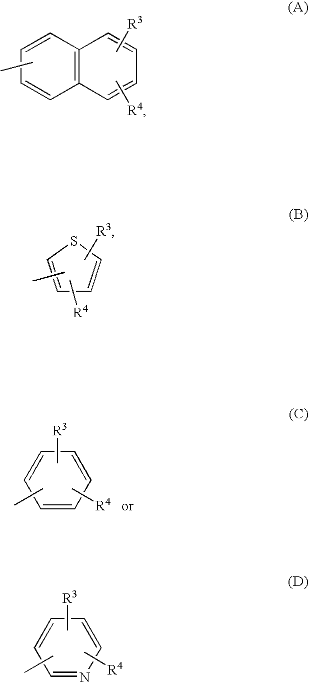

wherein
R³ and R⁴ represent each independently hydrogen, halogen, aryl, alkoxy, carboxy, hydroxy, alkoxyalkyl, alkoxycarbonyl, cyano, trifluoromethyl, alkanoyl, alkanoylamino, trifluoromethoxy, an optionally substituted aryl group, and
R_{B} is hydrogen, alkyl, alkanoyl, hydroxyalkyl, alkoxyalkyl, alkoxycarbonyl, alkoxycarbonylalkyl, aminoalkyl, mono- or dialkylaminoalkyl or Het-alkyl, where Het is as defined above;
provided that
   (i) when R_{C} is dialkylamino, then R_{B} is not hydrogen or alkyl;
   (ii) when R_{A} is a group of formula (C), where R³ is hydrogen and R₄ is methoxy, then R_{C} is not Het—CO—N(R′)—; and
   (iii) when R_{A} is a group of formula (C), where R³ and R⁴ are hydrogen or halogen, then R_{C} is not nitro.

Typical sulphonamide compounds of the present invention are shown in Table 2.

TABLE 2

| | Compound |
|---|---|
| | no. |
| 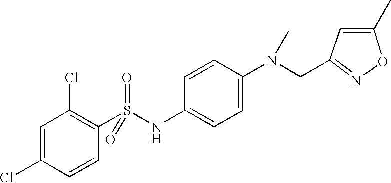 | 329 |
| 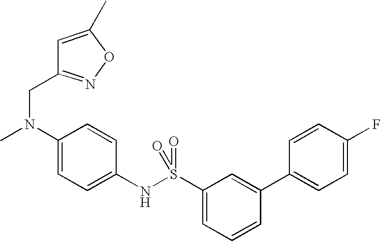 | 343 |
| 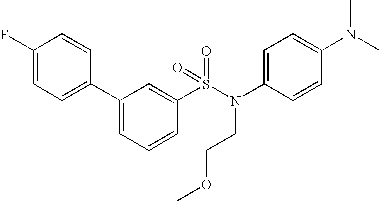 | 353 |
| 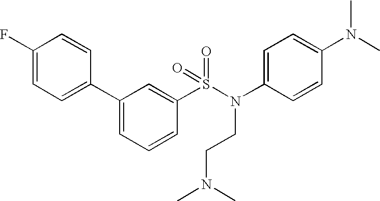 | 354 |
| 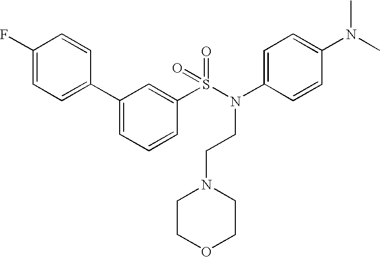 | 355 |
| 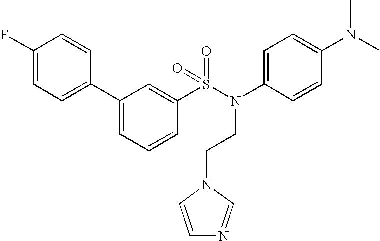 | 358 |
| 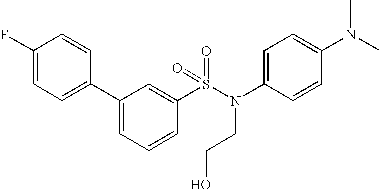 | 359 |
| 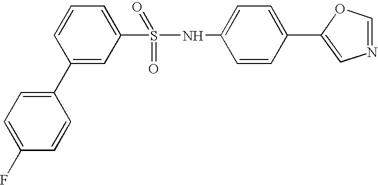 | 378 |
| 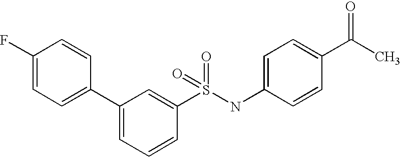 | 383 |
| 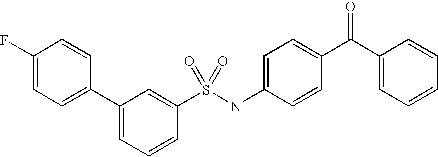 | 384 |
| 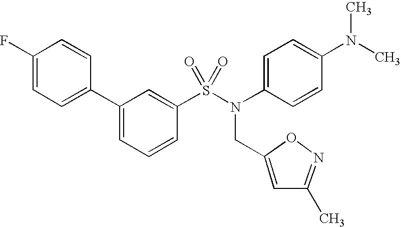 | 386 |
| 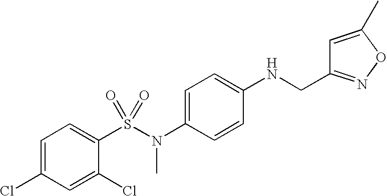 | 389 |
| 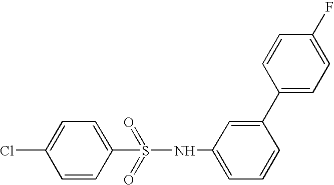 | 398 |
| 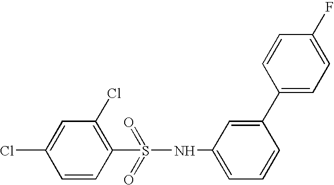 | 403 |
| 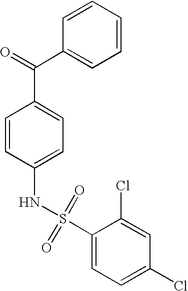 | 416 |
| 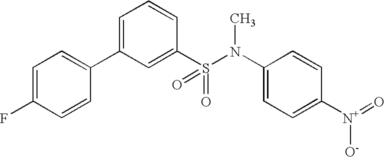 | 428 |
| 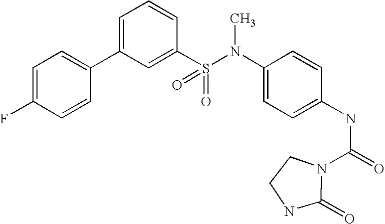 | 430 |
| 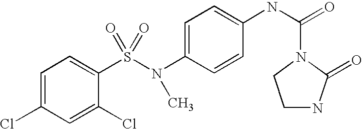 | 431 |
| 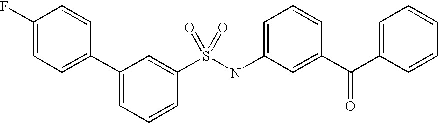 | 432 |
| 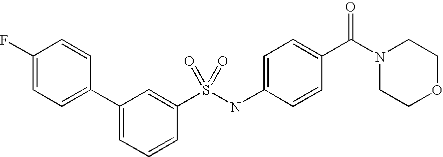 | 433 |
| 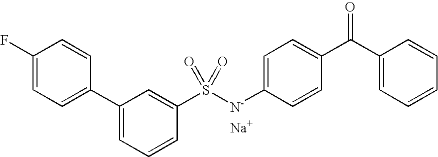 | 434 |
| 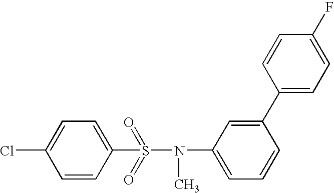 | 436 |
| 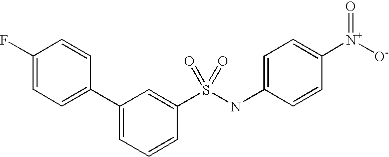 | 440 |
| 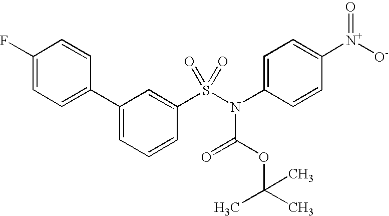 | 441 |
| 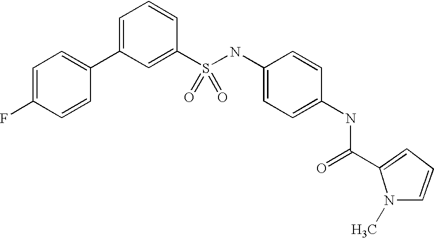 | 442 |
| 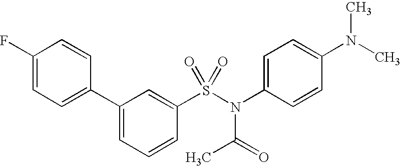 | 445 |
| 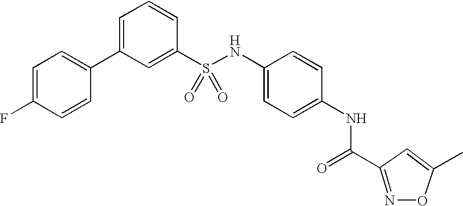 | 443 |
| 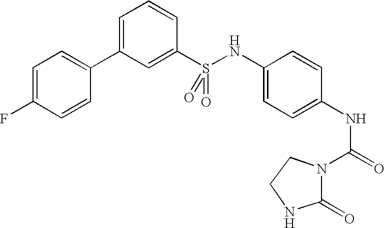 | 447 |
| 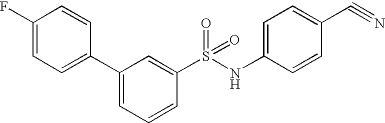 | 448 |
| 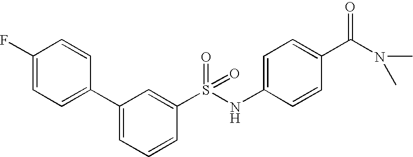 | 451 |
| 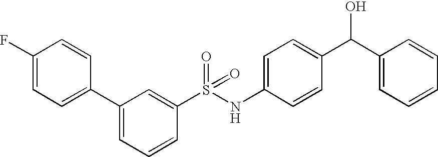 | 452 |
| 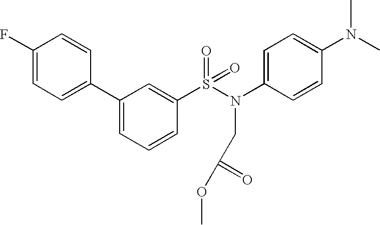 | 454 |
| 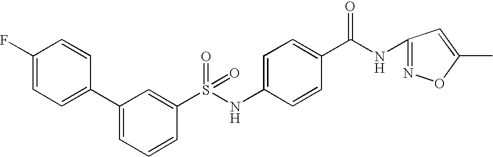 | 456 |
| 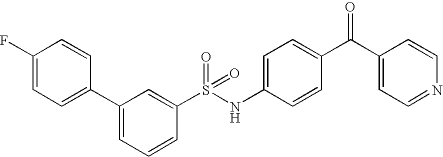 | 457 |
| 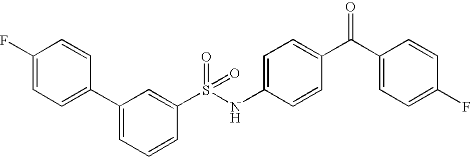 | 458 |

Specific examples of preferred compounds are:

4′-fluoro-biphenyl-3-sulfonic acid (4-benzoyl-phenyl)-amide,

4′-fluoro-biphenyl-3-sulfonic acid (3-benzoyl-phenyl)-amide,

4′-fluoro-biphenyl-3-sulfonic acid (α-hydroxybenzyl-phenyl)-amide,

2-oxo-imidazolidine-1-carboxylic acid{4-[(4′-fluoro-biphenyl-3-sulfonyl)-methyl-amino]-phenyl}-amide.

The present invention thus provides novel integrin-inhibitors, that fulfil the key interactions required by the MIDAS amino acid residues as described in the refined in silico model. Preferred integrin inhibitors are sulphonamide derivatives listed in Table 2 and the tetracyclic polyketides listed in Table 3.

The present invention provides the use of such integrin-modulators for the manufacture of a medicament for use in the treatment of diseases related to thrombosis, cancer, fibrosis and inflammation.

The compounds of the present invention are potent collagen receptor modulators and useful for inhibiting or preventing the adhesion of cells on collagen or the migration and invasion of cells through collagen, in vivo or in vitro. The now described compounds inhibit the migration of malignant cells and are thus useful for treating diseases such as cancers, including prostate, gastric, pancreatic and ovary cancer, and melanoma, especially where α2β1 integrin dependent cell adhesionlinvasion/migration may contribute to the malignant mechanism, cancer invasion and metastasis or angiogenesis.

The compounds of the invention also inhibit adhesion of platelets to collagen and collagen-induced platelet aggregation. Thus, the compounds of the invention are useful for treating patients in need of preventive or ameliorative treatment of thromboembolic conditions i.e. diseases that are characterized by a need to prevent adhesion of platelets to collagen and collagen-induced platelet aggregation, for example treatment and prevention of stroke, myocardial infarction, unstable angina pectoris, diabetic retinopathy or retinal vein occlusion. The compounds of the present invention are further useful as medicaments for treating patients with disorders characterized by inflammatory processes, such as inflammation, fibrosis and bone fractures.

To conclude, the present invention provides a successful strategy to design collagen receptor integrin inhibitors targeted to MIDAS in α I-domains. Aromatic polyketides and sulfonamides fulfil the criteria for potential blockers of collagen receptor α I-domains and they also prevent cell adhesion to collagen, but other compound that fulfil the criteria defined by the refined in silico model are considered compounds according to the present invention.

The following examples are given to illustrate the invention but are not intended to limit the scope of the invention.

### EXAMPLES

### Example 1

### Tetracycline Biosynthesis

A library of tetracycline compounds was produced by fermentation of a mutant Streptomyces strain. The fermentation was performed as a 5 litre batch for six days in E1 medium at 30° C., aeration 5 l/h by stirring 280 rpm.

The metabolites were collected from the cell fraction by methanol extraction, whereafter the compounds were extracted with dichloromethane, analyzed and evaporated.

A preliminary purification of the compounds was performed by two chromatographic treatments followed by precipitation. The purification was monitored by Thin Layer Chromatography (TLC). The first chromatographic separation was done in a column containing silica in chloroform:methanol:acetic acid. The fractions were eluted utilizing 2% methanol. The combined fractions were further purified in a silica column eluted with toluene:MeOH:HCOOH.

The collected fractions were combined, diluted in a small amount of chloroform and precipitated with hexane. The tetracyclic compounds were concentrated in the hexane phase. The hexane was evaporated and this fraction was used as starting material in further purification.

The fractions received from the preliminary purification were further purified with oxalate treated silica column, eluted with 40% hexane in chloroform. The fractions containing tetracyclic compounds were further purified in a preparative C18 HPLC column, with acetonitrile:water:formic acid. The pure fractions were combined, dissolved in chloroform and evaporated.

Compounds thus received and further tested were methyl 2-ethyl-2,5,7,12-tetrahydroxy-4,6,11-trioxo-1,2,3-trihydronaphthacene-carboxylate (L3007), methyl 2-ethyl-4,5,7,12-tetrahydroxy-6,11- dioxonaphthacenecarboxylate (L3008), methyl 4,5,7,12-tetrahydroxy-2-methylethyl)-6,11- dioxonaphthacenecarboxylate (L3009) and methyl 2-ethyl-4,5,7-trihydroxy-6,11 dioxonaphthacenecarboxylate (L3015). The structures of the compounds are given in Table 3.

TABLE 3

| | |
|---|---|
| 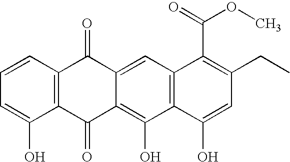 | L3015 |
| 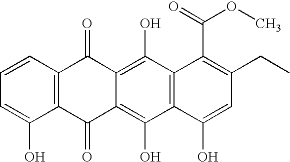 | L3008 |
| 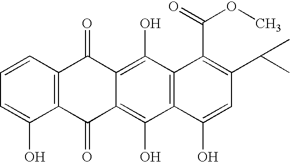 | L3009 |
| 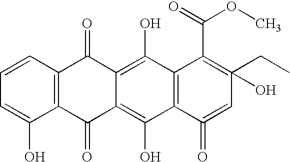 | L3007 |

### Example 2

### Human Recombinant Integrin I-Domains

Cloning of human integrin α I-domains- cDNAs encoding α1I and α2 I-domains were generated by PCR as described earlier using human integrin α1 and α2 cDNAs as templates. Vectors pGEX-4T-3 and pGEX-2T (Pharmacia) were used to generate recombinant glutathione S-transferase (GST) fusion proteins of human α1I and α2 I-domains, respectively The α10 I-domain cDNA was generated by RT-PCR from .RNA isolated from KHOS-240 cells (Human Caucasian osteosarcoma). Total cellular RNA was isolated by using RNeasy Mini Kit (Qiagen). RT-PCR was done using the Gene Amp PCR Kit (Perkin Elmer). Details for the cloning are described earlier (Tulla et al., 2001). The amplified α10 I-domain cDNA was digested along with pGEX-2T expression vector (Amersham Pharmacia Biotech) using the BamHI and EcoRI restriction enzymes (Promega). To the pGEX-2T vector the α10 cDNA was ligated with the SureClone Ligation Kit (Amersham Pharmacia Biotech). The construct was transformed into the E. coli BL21 strain for the production. The DNA sequence of the construct was checked with DNA sequencing and compared to the published α10 DNA sequence (Camper et al., 1998). Human integrin α11 cDNA was used as a template when α11 I-domain was generated by PCR.

Expression and purification of α I-domains—Competent E. coli BL21 cells were transformed with the plasmids for protein production. 500 ml LB medium (Biokar) containing 100 μg/ml ampicillin was inoculated with 50 ml overnight culture of wild-type or mutant BL21/pal and the cultures were grown at 37° C. until the O.D.600 of the suspension reached 0.6-1.0. Cells were induced with IPTG and allowed to grow for an additional 4-6 h typically at room temperature before harvesting by centrifugation. Pelleted cells were resuspended in PBS (pH 7.4), then lysed by sonication followed by addition of Triton X-100 to a final concentration of 2%. After incubation for 30 min on ice, suspensions were centrifuged, and supernatants were pooled. Glutathione Sepharose® 4B (Amersham Pharmacia Biotech) was added to the lysate, which was incubated at room temperature for 30 min with gentle agitation. The lysate was then centrifuged, the supernatant was removed, and Glutathione Sepharose® 4B with bound fusion protein was transferred into disposable chromatography columns (Bio-Rad). The columns were washed with PBS, and fusion proteins were eluted using 30 mM reduced glutathione.

Purified recombinant and glutathione-tagged α I-domains were analysed by SDS and native polyacrylamide gel electrophoresis (PAGE). Protein concentrations were measured with Bradford's method (Bradford, 1976). The recombinant α1 I-domain produced was 227 amino acids in length, corresponding to amino acids 123-338 of the whole α1 integrin, while the α2 I-domain was 223 amino acids long which corresponded to amino acids 124-339 of the whole α2 integrin. The carboxyl termini of the α1I and α2 I-domains contained ten and six non-integrin amino acids, respectively (Käpylä et al., 2000, Tulla et al., 2001). Recombinant α10 I-domain produced was 197 amino acids in length, corresponding to amino acids 141-337 of the whole α10 integrin. The amino terminal contained two non-integrin residues and the carboxy terminal of α10I contained six non-integrin amino acids (Tulla et al., 2001). Recombinant α11 I-domain contains totally 204 amino acids: in the amino terminal there are two extra residues before α11I, residues 159-354, in the carboxy terminal there are six extra amino acids. Recombinant α11 I-domain contains some GST as an impurity due to the endogenous protease activity during expression and purification (Zhang et al., 2003). Recombinant α I-domains were used as GST-fusion proteins for collagen binding experiments.

Site-directed mutagenesis—Site-directed mutation of the α I-domains cDNA in a pGEX-2T or pGEX4T-3 vector was made using PCR according to Stratagene's QuickChange Mutagenesis Kit instructions. The presence of mutations was checked by DNA sequencing. Mutant constructs were then transformed into E. coli strain BL21 for production of recombinant protein (Käpylä et al., 2000; Tulla et al., 2001).

### Example 3

### Generation of α2 I-Domain Mutants

Site-specific mutations in α2 I-domain were made using the Stratagene QuickChange mutagenesis kit following the manufacturer's instructions. PCR primers having the desired mutations for both DNA-strands were designed. PCR was performed using Pfu polymerase (Stratagene), which makes at 68° C. one copy of the whole GEX-2T vector (Amersham Pharmacia Biotech) containing the α2 I-domain sequence. The PCR was digested with Dpnl, which cuts only methylated DNA. After that, PCR product DNA strands having the desired mutation were paired.

### Example 4

### Alpha—I-Domain Binding Assay

Solid-phase binding assay for α1-domains—The coating of a 96-well high binding microtiter plate (Nune) was done by exposure to 0.1 ml of PBS containing 5 μg/cm2 (15 μg/ml) collagens or 20 μg/ml triple-helical peptides overnight at +4° C. Blank wells were coated with 1:1 solution of 0.1 ml Delfia® Diluent II (Wallac) and PBS. Residual protein absorption sites on all wells were blocked with 1:1 solution of 0.1 ml Delfia® Diluent II (Wallac) and PBS. Recombinant proteins (αI-GST) were added to the coated wells at a desired concentration in Delfia® Assay Buffer and incubated for 1 h at room temperature. Europium-labelled anti-GST antibody (Wallac) was then added (typically 1:1000), and the mixture was incubated for 1 h at room temperature. All incubations mentioned above were done in the presence of 2 mM MgCl2. Delfia® enhancement solution (Wallac) was added to each well and the Europium signal was measured by time-resolved fluorometry (Victor2 multilabel counter, Wallac). At least three parallel wells were analyzed. In some cases some what modified solid-phase assay was used and it was performed according Tulla et al, 2001. It uses anti-GST and Europium-labelled protein G instead of Europium-labelled anti-GST antibody.

### Example 5

### Cell Adhesion Assay

Chinese Hamster Ovary (CHO) cell clone expressing wild type α2 integrin was used in cell adhesion assay. Cells were suspended in serum free medium containing 0.1 mg/ml cycloheximide (Sigma) and the compounds were preincubated with the cells prior to transfer to the wells. Cells (150000/well) were allowed to attach on collagen type I coated wells (in the presence and absence of inhibitor compounds) for 2 h at +37° C. and after that non-adherent cells were removed. Fresh serum free medium was added and the living cells were detected using a cell viability kit (Roche) according to the manufacturers protocol.

### Example 6

### Molecular Modelling

The binding modes for the discovered tetracyclic polyketide and sulphonamide α2 integrin I-domain modulators were unknown prior to this work. We used standard and proprietary molecular modelling tools in combination with experimental evidence to identify the bioactive conformations of tetracyclic polyketides and sulphonamides in complex with the α2 integrin ligand binding (MIDAS) site. The structure of the MIDAS was modelled using BODIL. The modelled MIDAS structure was utilized to superpose the structurally and functionally diverse modulators. In the modelling simulations we explored the conformational space of the modulators, while taking into account the chemical and structural features of the MIDAS. This procedure provided preferred binding conformations for each ligand structure. The information was then used to derive the structural rules for interactions that are required from small molecules that modulate collagen binding through α2 integrin MIDAS.

The crystal structures of open (PDB ID: 1dzi) and closed forms (PDB ID: 1aox) of I-domain used as a starting point in molecular modelling were retrieved from the Protein Data Bank. Amino acid side chain conformations were altered in the BODIL software to create an ensemble of protein conformations using the BODIL rotamer libraries. Key structural waters coordinated in the MIDAS were included in the docking simulation as part the crystal structure. All hydrogens of the protein structures and of the water molecule were added using Sybyl 6.9.1 (rotate). Docking was made using FlexX in SY-BYL 6.9.1. and automated rotation-translation procedure in BODIL, which docked unconstrained ligand conformations produced using Comfort/Concord in SY-BYL 6.9.1. In addition to FlexX scoring, for each docked ligand structure the free energy of binding was evaluated with Xscore (Wang et al., 2002).

### Example 7

### Inhibition of Collagen Binding by Compounds Identified in Silico

The tetracyclic Streptomyces compounds synthesised in Example 1, where screened for inhibition of collagen binding to α1 and α2 I-domains, using the α I-domain assay described in Example 4. Tetracyclic polyketide, L3015, was a relatively potent inhibitor of α2 I-domain binding to type I collagen. It showed dose dependent inhibition of α2 I-domain binding to type I collagen (about 50% inhibition at 0.03 mM concentration; FIG. 6A). L3015 could inhibit the binding of both α1I and α2 I-domains to type I and type IV collagen (FIG. 6B).

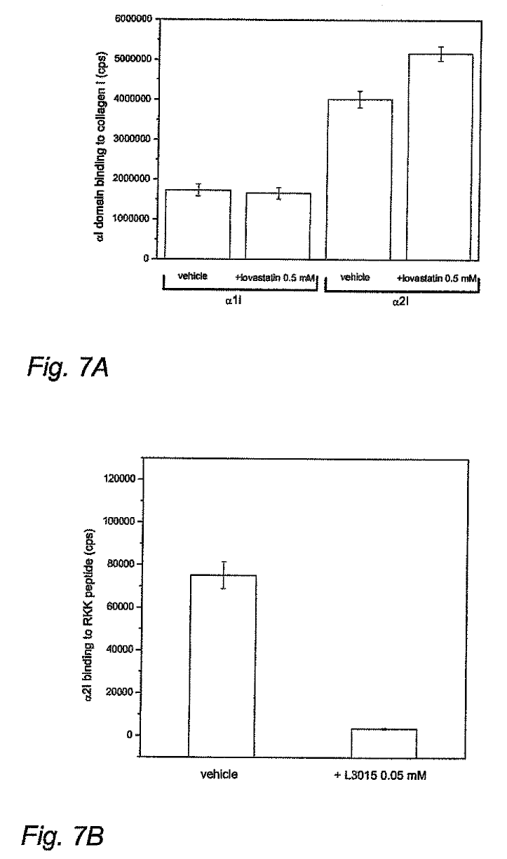

RKK-peptides are known to bind to MIDAS of α2 I-domain (Ivaska et al., 1999). Integrin α2 I-domain binding to RKK-peptide in the presence of L3015 was tested in the europium-labelled protein G assay described in Example 4. The results show that L3015 can displace RKK peptide at MIDAS (FIG. 7B).

Furthermore α1I and α2 I-domain binding to collagen I in the presence of lovastatin was tested in the europium-labelled anti-GST assay described in Example 4. Lovastatin is an allosteric inhibitor of leukocyte integrin α I-domains (e.g. αL I-domain), and the binding site of lovastatin represents an optional binding site for possible modulators. However lovastatin was shown to have no effect on α I-domain binding to collagen type I (FIG. 7A). These biological tests gave further evidence that collagen receptor α I-domains are inhibited by direct blocking of the MIDAS surface by tetracyclic polyketide.

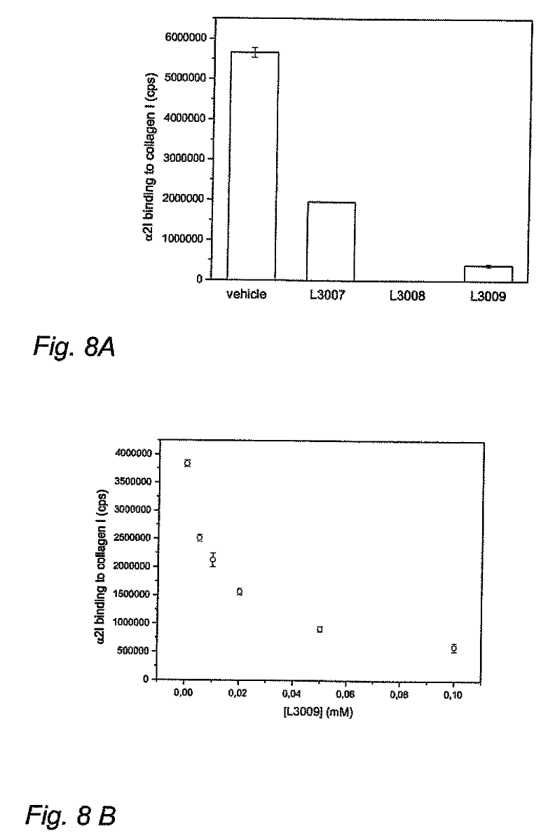

Based on the utilization of 3D model other compounds from the polyketide family were tested. The compounds were tested in the europium-labelled anti-GST assay described in Example 4. Tetracyclic polyketides L3007, L3008, and L3009 could inhibit α2 I-domain binding to type I collagen (FIG. 8A). The dose dependent inhibition effect of one of the most active structure, L3009, is shown in FIG. 8B.

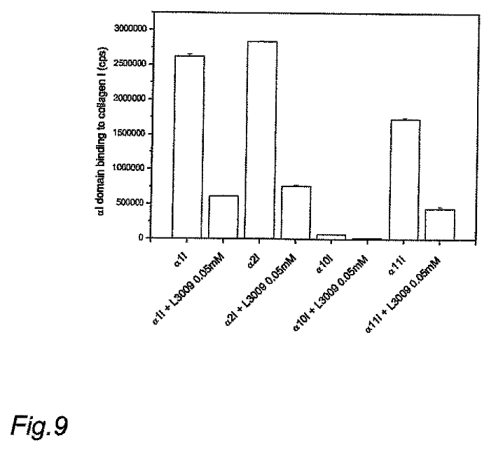

The inhibitory effect of L3009 was tested with all collagen binding integrin α I-domains, α1I, α2I, α10I and α11I as described in Example 4. L3009 could inhibit the collagen I binding of all four α I-domains at 0.05 mM concentration (FIG. 9).

The most potent compound, L3009, was tested further in the functional cell adhesion assay described in Example 5 in order to study the function of integrin heterodimers on cell surface. For this purpose CHO cells were transfected to express α2β1 integrin on their surface as their only collagen receptor.

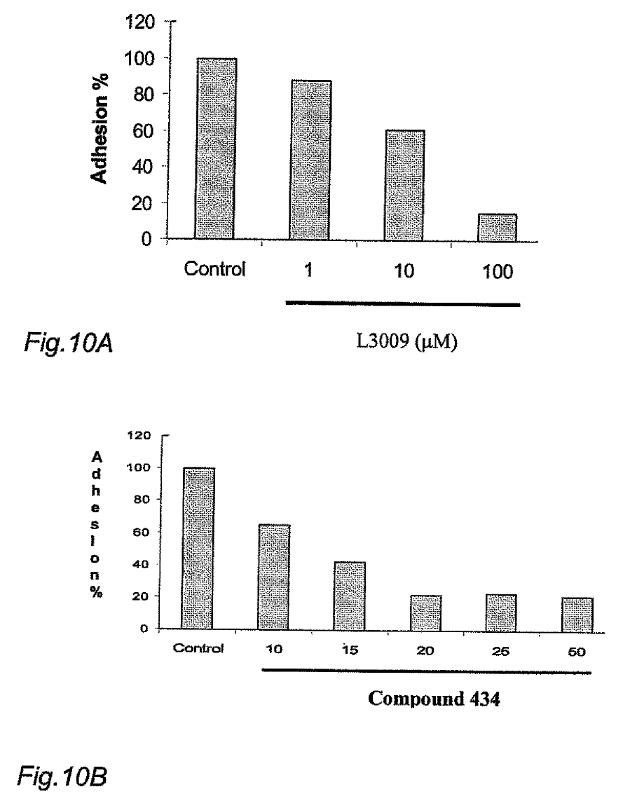

L3009 was a potent inhibitor of cell adhesion to type I collagen, with EC50 value of about 20 μM (FIG. 10A).

The in silico model was utilized to identify novel collagen receptor modulators. Sulphonamide derivatives, compound 434 and compound 161, are examples of novel molecules identified with the method. Compound 434 was tested in the functional cell adhesion test described in Example 5. FIG. 10B and Table 4 show that compound 434 is a potent inhibitor of cell adhesion to collagen type I.

TABLE 4

| | | | | EC50 |
|---|---|---|---|---|
| Com- | EC50, | | | in cell |
| pound | adhesion | Emax, adhesion | Inhibition % at | Invasion |
| number | (uM) | (at 100 uM; %) | 50 uM; adhesion | (uM) |
| 354 | 29 | 89 | 78 | nt |
| 358 | 30 | 74 | 71 | nt |
| 359 | 39 | 67 | 42 | nt |
| 383 | 39 | (—) | 26 | nt |
| 384 | 12 | 81 | 65 | 0.8 |
| 398 | 16 | (—) | 83 | nt |
| 403 | 37 | 86 | 56 | nt |
| 416 | 42 | 79 | 44 | nt |
| 430 | 14 | 59 | 53 | 12 |
| 432 | 15 | 79 | 72 | 1 |
| 434 | 9.1 | 78 | 78 | 0.8 |
| (salt | | | | |
| of 384) | | | | |
| 440 | 8.7 | 78 | 59 | 8.3 |
| 442 | (—) | 22 | 21 | nt |
| 448 | 38 | 74 | 58 | 27 |
| 452 | 9.4 | (—) | 83 | 0.8 |

nt = not tested

### Example 8

### Integrin α2 I-Domain Mutations

To confirm the role of α2β1 integrin α2 I-domain amino acids in modulator binding, site-directed mutagenesis approach was used. The selected amino acid mutations were made as described in Example 3. Single amino acids in α2 I-domain region were mutated and tested in adhesion experiments using CHO expressing mutated α2β1 integrin; wild-type α2β1 expressing cells were used as a control. The cell adhesion experiments were done as described in Example 5. Results of the studies revealed three amino acids of α2 I-domain to be important for the inhibitory function of L3008: tyrosine 285, leucine 286 and leucine 296. Mutation in these amino acids significantly decreased the inhibitory effect of tetracyclic polyketide L3008, sulfonamide compound 161 and sulphonamide compound 434 in the adhesion of CHO cells expressing α2β1 to collagen I (data not shown).

### Example 9

### Cell Invasion Assay to Demonstrate the Anti-Cancer Potential of the Inhibitors in Vitro

The ability to interact with extracellular matrix basement membranes is essential for the malignant cancer cell phenotype and cancer spread. α2β1 levels are known to be upregulated in tumorigenic cells. The overexpression regulates cell adhesion and migration to and invasion through the extracellular matrix. By blocking the interaction between extracellular matrix components like collagen and α2β1 it is possible to block cancer cell migration and invasion in vitro. Prostate cancer cells (PC-3) expressing α2β1 endogenously were used to test the in vitro anticancer potential of the modulators of the present invention.

Experimental procedure. Invasion of PC-3 cells (CRL-1435, ATCC) through Matrigel was studied using BD Biocoat invasion inserts (BD Biosciences). Inserts were stored at −20° C. Before the experiments inserts were allowed to adjust to the room temperature. 500 μl of serum free media (Ham's F12K medium, 2 mM L-glutamine, 1.5 g/l sodium bicarbonate) was added into the inserts and allowed to rehydrate at 37° C. in cell incubator for two hours. The remaining media was aspirated. PC-3 cells were detached, pelleted and suspended into serum free media (50,000 cells/500 μl ). 300 μl of cell suspension was added into the insert in the absence (control) or presence of the inhibitor according to the present invention. Inserts were placed on the 24-well plates; each well containing 700 μl of cell culture media with 3% of fetal bovine serum as chemo-attractant. Cells were allowed to invade for 72 hours at 37° C. in cell incubator. Inserts were washed with 700 μl PBS, and fixed with 4% paraformaldehyde for 10 minutes. Paraformaldehyde was aspirated and cells were washed with 700 μl of PBS and inserts were stained by incubation with hematoxylin for 1 minute. The stain was removed by washing the inserts with 700 μl of PBS. Inserts were allowed to dry. Fixed invaded cells were calculated under the microscope. Invasion % was calculated as a comparison to the control.

This cell invasion assay was used as an in vitro cancer metastasis model. The sulphonamide molecules were shown to inhibit tumour cell invasion in vitro (Table 4). Some structures inhibit invasion even with submicromolar concentrations.

### Example 10

### Use of a Platelet function analyzer PFA-100 to demonstrate the antithrombotic potential of the α2β1 Modulators

A platelet function analyzer PFA-100 was used to demonstrate the possible antithrombotic effects of α2β1 modulators. The PFA-100 is a high shear-inducing device that simulates primary haemostasis after injury of a small vessel. The system comprises a test-cartridge containing a biologically active membrane coated with collagen plus epinephrine. An anticoaculated whole blood sample was run through a capillary under a constant vacuum. The platelet agonist (epinephrine) on the membrane and the high shear rate resulted in activation of platelet aggregation, leading to occlusion of the aperture with a stable platelet plug. The time required to obtain full occlusion of the aperture was designated as the “closure time”. Each hit compound was added to the whole blood sample and the closure time was measured with PFA-100. If the closure time was increased when compared to the control sample the hit compound was suggested to have antithrombotic activity.

Experimental procedure. Blood was collected from a donor via venipuncture into evacuated blood collection tubes containing 3.2% buffered sodium citrate as anticoagulant. Blood was aliquoted into 15 mL tubes and treated with either inhibitory compounds or controls (DMSO). Samples were kept at room temperature with rotation for 10 minutes and after that the closure time of the blood was measured.

Acquisitions resulting in a closure time exceeding the range of measurement of the instrument (>300 seconds) were assigned a value of 300 seconds. Mean and standard deviations were calculated for each treatment. Student's t-test was applied to the resultant data.

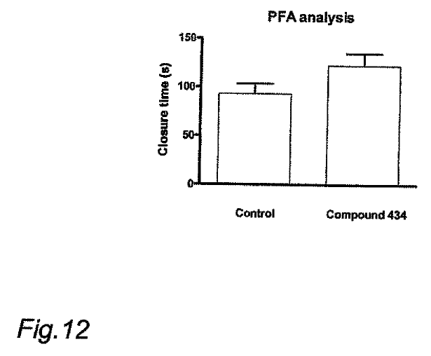

Compound 434 was shown to increase the closure time of the blood (FIG. 12).

What is claimed is:

1. A refined in silico model of the MIDAS of α2β1integrin I-domain, characterized by the amino acid coordinates Asp151, Ser153, Ser155, Thr221, Asp254, Tyr285, Leu286 and Leu296.

2. The model according to claim 1, characterized by the amino acid coordinates Asn154, Gly218, Asp219, Gly255, Glu256, Asn289, Leu291 and Asp292.

3. The model according to claim 2, characterized by the amino acid coordinates shown in Table 1.

4. The model according to any one of claims 1 to 3, characterized by key water molecules W514, W699, W701, W700, W668, W597, W644 and W506.

5. A method of identifying compounds modulating an α2β1integrin, comprising the steps of:
(a) applying an algorithm for 3-dimensional molecular modelling to the atomic coordinates of an α2β1 I-domain-containing integrin to determine the spatial coordinates of the metal ion dependent adhesion site (MIDAS) of said integrin; and
(b) in silico screening stored spatial coordinates of a set of candidate compounds against said spatial coordinates determined in step (a) to identify compounds that can bind to the MIDAS of said integrin.

6. The method according to claim 5, fuirther comprising the steps of:
(c) providing a fragment of an integrin α2 I-domain, which fragment contains the amino acid residues used in said model;
(d) bringing said fragment into contact with said candidate modulator; and
(e) determining the ability of the peptide fragment to bind with said potential inhibitor.

7. The method according to any one of claim 5 and 6, wherein said compounds are integrin inhibitors.

8. An α2β1 I-domain-containing integrin modulating compound, identified or obtained by the method according to any one of claims 5 and 6.

9. The compound according to claim 8, having the general formula (I) 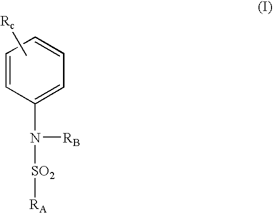
where
R_{C} is selected from a group consisting of dialkylamino, NO₂, CN, aminocarbonyl, monoalkylaminocarbonyl, dialkylaminocarbonyl, alkanoyl, oxazol-2-yl, oxazolylaminocarbonyl, aryl, aroyl, aryl-CH(OH)—, arylaminocarbonyl, furanyl, where the aryl, aroyl and furanyl moieties may be substituted, guanidinyl-(CH₂)_{z}—N(R′)—, Het-(CH₂)_{z}—N(R′)—, Het—CO—N(R′)—, Het—CH(OH)— and Het—CO—, where Het is an optionally substituted 4-6-membered heterocyclic ring containing one or more heteroatoms slected from N, O and S, R′ is hydrogen or alkyl, and z is an integer 1 to 5;
R_{A} is a group having the formula 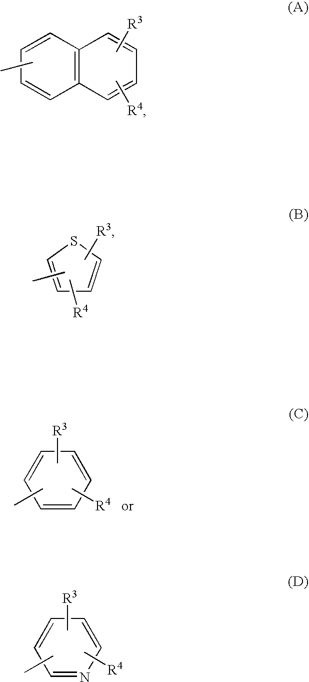
wherein
R³ and R⁴ represent each independently hydrogen, halogen, aryl, alkoxy, carboxy, hydroxy, alkoxyalkyl, alkoxycarbonyl, cyano, trifluoromethyl, alkanoyl, alkanoylamino, trifluoromethoxy, an optionally substituted aryl group, and
R_{B} is hydrogen, alkyl, alkanoyl, hydroxyalkyl, alkoxyalkyl, alkoxycarbonyl, alkoxycarbonylalkyl, aminoalkyl, mono- or dialkylaminoalkyl or Het-alkyl, where Het is as defined above;
provided that
(i) when R_{C} is dialkylaamino, then R_{B} is not hydrogen or alkyl;
(ii) when R_{A} is a group of formula (C), where R³ is hydrogen and R₄ is methoxy, then R_{C} is not Het—CO—N(R′)—; and
(iii) when R_{A} is a group of formula (C), where R³ and R⁴ are hydrogen or halogen, then R_{C} is not nitro.

10. The compound according to claim 8, which is an integrin inhibitor.

11. The compound according to claim 9, which is 4′-fluoro-biphenyl-3-sulfonic acid (4-benzoyl-phenyl)-amide.

12. The compound according to claim 9, which is 4′-fluoro-biphenyl-3-sulfonic acid (3-benzoyl-phenyl)-amide.

13. The compound according to claim 9, which is 4′-fluoro-biphenyl-3-sulfonic acid (α-hydroxybenzyl-phenyl)-amide.

14. The compound according to claim 9, which is 2 oxo imidazolidine 1 carboxylic acid {4-[(4′-fluoro-biphenyl-3-sulfonyl)-methyl-amino]-phenyl}-amide.

15. The compound according to claim 9, which is a tetracyclic polyketide.

16. The compound according to claim 15, which has the formula methyl 2-ethyl-2,5,7,12-tetrahydroxy-4,6,11-trioxo-1,2,3-trihydronaphthacenecarboxylate.

17. The compound according to claim 15, which has the formula methyl 2-ethyl-4,5,7,12-tetrahydroxy-6,11-dioxonaphthacenecarboxylate.

18. The compound according to claim 15, which has the formula methyl 4,5,7,12-tetrahydroxy-2-(methylethyl)-6,11-dioxonaphthacenecarboxylate.

19. The compound according to claim 15, which has the formula methyl 2-ethyl-4,5,7-trihydroxy-6,11-dioxonaphthacenecarboxylate.

20-24. (canceled)

25. A method of treating a thrombosis, cancer, fibrosis or inflammation by administering to a patient in need of such treatment an effective amount of a compound according to any one of claims 8 to 9.

26. The method according to claim 25 for the manufacture of a pharmaceutical composition for the treatment of prostate, gastric, pancreatic or ovary cancer, or melanoma and prevention of cancer angiogenesis.

27. The method according to claim 26 for the manufacture of a pharmaceutical composition for the prevention or treatment of metastases.

28. The method according to claim 25 for the manufacture of a pharmaceutical composition for the treatment of stroke, myocardial infarction, diabetic retinopathy or retinal vein occlusion.

29. The method according to claim 25 for the manufacture of a pharmaceutical composition for the treatment of inflammatory diseases associated with fibrosis and bone fractures.
